(19) United States
(12) Patent Application Publication
(10) Pub. No.: US 2025/0311943 A1
(43) Pub. Date: Oct. 9, 2025

(54) DEVICE AND METHOD FOR STANDARDIZING THE AXIAL ORIENTATION AND POSITION OF KINEMATICS DATA WITH REGARD TO A BODY JOINT OF A PATIENT

(71) Applicant: Aesculap AG, Tuttlingen (DE)

(72) Inventors: Michael Utz, Tuttlingen (DE); Ariana Ortigas Vasquez, Konstanz (DE); Adrian Sauer, Oberkirch (DE); Allan Maas, Konstanz (DE); William R. Taylor, Zuerich (CH)

(21) Appl. No.: 18/865,210

(22) PCT Filed: May 12, 2023
(86) PCT No.: PCT/EP2023/062780

§ 371 Date: Nov. 12, 2024

(30) Foreign Application Priority Data
May 13, 2022 (DE) 10 2022 112 117.8
Oct. 5, 2022 (DE) 10 2022 125 697.9

(51) Int. Cl. A61B 5/11; A61B 5/00; A61B 34/10

(52) U.S. Cl. CPC A61B 5/112; A61B 5/1121; A61B 5/1126; A61B 5/7271; A61B 34/10; A61B 2034/108; A61B 2562/0219

(57) ABSTRACT

A system for acquiring, standardizing and evaluating biomechanical kinematics data of a joint includes a device for acquiring biomechanical kinematics of the joint and processing it in a computer-readable manner. A display device displays visual output to a user. A control unit processes the kinematics and determines a progression of kinematics parameters of the joint with respect to a first joint element with a first joint coordinate system and with respect to a second joint element with a second joint coordinate system. A standardization unit determines, via a predetermined target optimization, a transformation for adjusting an orientation and/or position of the first and/or second joint coordinate system to carry out a standardization, and output an evaluation via the display device. A method is used to acquire, standardize and evaluate biomechanical kinematics data of a joint, and can be executed by a computer with a computer-readable storage medium.

### CROSS-REFERENCE TO RELATED APPLICATIONS

This application is the United States national stage entry of International Application No. PCT/EP2023/062780, filed on May 12, 2023, and claims priority to German Application No. 10 2022 112 117.8, filed on May 13, 2022, and claims priority to German Application No. 10 2022 125 697.9, filed on Oct. 5, 2022. The contents of International Application No. PCT/EP2023/062780, German Application No. 10 2022 112 117.8, and German Application No. 10 2022 125 697.9 are incorporated by reference herein in their entireties.

### FIELD

The present disclosure relates to a standard kinematics evaluation system for acquiring, standardizing and evaluating biomechanical kinematics data of the (selected) joint of a patient, comprising: at least one acquisition device adapted to detect (record) the biomechanical kinematics of a patient's joint by means of sensors, and provide them in a computer-readable form; and a visual display device, in particular a operating room monitor, for visual output to a user. In addition, the present disclosure relates to a standardization method and a computer-readable storage medium.

### BACKGROUND

In kinematic analysis of moving bodies or segments, the position/pose, i.e. the alignment and position, but in particular already the alignment itself of the underlying reference system of each segment involved has considerable influence on the magnitude and characteristics/properties of the resulting (kinematic) curve, in particular waveforms, of three measured rotations corresponding to a specific joint connecting these segments, and possibly additionally measured translations in three spatial directions. In the past, this was ascribed to crosstalk effects.

Crosstalk results from incorrect alignment and, moreover, preferably an incorrect positioning of the axes of the coordinate system, so that a rotation in one plane is partially perceived as a rotation in other planes.

Methods for minimizing crosstalk effects usually try to achieve this by minimizing one or more selected target functions for a particular activity, measurement system, or biomechanical model. Such minimization is described, for example, in Baker et al. 1999—A new approach to determine the hip rotation profile from clinical gait analysis data, Rivest 2005—A correction for axis misalignment in the joint angle curves representing knee movement in gait analysis, Baudet et al. 2014—Cross-Talk Correction Method for Knee Kinematics in Gait Analysis Using Principal Component Analysis (PCA): A New Proposal.

These methods have a significant disadvantage to the effect that, for at least one of the data sources to be compared, an alignment, and moreover preferably a positioning, of the underlying reference frames relative to the segments that they represent, is unknown.

The same applies to methods that attempt to compare the extent to which data from different sources (e.g., different biomechanical models, different laboratories, different marker sets, and the like) are concerned by crosstalk effects. In those approaches crosstalk so far was quantified using at least one representative parameter (e.g. a measure of determination between flexion/extension and abduction/adduction), and then it was concluded that a lower value of such a parameter could be interpreted as a sign of lower crosstalk effects (Kainz et al. 2016—Joint kinematic calculation based on clinical direct kinematic versus inverse kinematic gait models).

However, prior to making such a comparison, all data sets absolutely have to be made comparable and standardized to a certain extent. The reason for this is that any comparison of non-standardized data is to some extent invalid, since it is unclear whether the displayed kinematic differences in fact reflect different motion patterns or merely are a result of different axis positions, especially alignment.

A large part of the existing literature that addresses the use of crosstalk minimization methods in rotational kinematic analysis attempts to justify the appropriate choice of parameters to be optimized (or minimized) by referring to prior research on so-called “physiological” characteristics of the motion.

Such reasoning and logic could be considered as incorrect since any experiment that seeks to measure say the “physiological” range of rotation of a knee joint in the transverse and frontal planes during flexion (in order to know to which values these rotations should be optimized in a crosstalk minimization algorithm), will necessarily require the definition of a specific reference frame in a particular alignment. The exact definition of such a frame (especially its orientation) will invariably affect the magnitude of the measured rotations. Therefore, the acquired “physiological” profile, which is often quoted as evidence why a chosen target function (or functions) is (are) valid, cannot in fact be reasonably used as a valid confirmation, as it itself is largely dependent on the frame definitions.

In considering two or more data sets, differences in the orientation of the corresponding joint axes may arise, inter alia, due to the enforcement of different kinematic constraints, the consideration of different underlying assumptions, and different modality algorithms for axis definition (e.g., landmark-based vs. function-based). As a result, the precise alignment of a joint reference frame relative to the connected rigid segments may vary from test to test. In considering, for example, two different tests that use the same optical motion detection system for data collection with the same test person performing the same activity, and where the axes are defined based on manual identification of the positions of bony reference points, using a special pointer, it is quite conceivable, in light of the possibility that one of the required reference points is identified in a slightly different position (if only a few millimeters) between these tests, that the sets of axes defined in each case are not perfectly aligned. If this scenario is widened to comparing data from different studies, differences in alignment may be due to a variable extent of a number of these factors. Without implementing a protocol that harmonizes those differences in alignment, conclusions about the presence of significant differences between groups are highly questionable.

Although methods exist to minimize the effects of crosstalk, these methods attempt to do so by minimizing one or more selected target functions. As mentioned earlier, these methods have a significant disadvantage when researchers wish to compare the quality of the resulting values with another data set for which either no crosstalk minimization or a different implementation of such was carried out.

The analysis of kinematic data of a patient's joint, in particular the knee, from different sources requires the use of standardized definitions and calculation conventions. While exact conversions exist for various underlying mathematical definitions, the normalization or standardization of the coordinate systems and their exact alignment is still challenging.

For example, if manual assessments are used for an analysis, such as the so-called anterior drawer test, these are very subjective. Alternatively, methods exist that use devices to measure joint laxity, which fix a reference joint segment and apply forces to another, connecting joint segment to thus induce and measure the resulting relative joint motion. However, neither of the above two approaches takes account of the effects of differences in the local positions and/or orientations of the segments, which leads to inconsistent results and disadvantageously to misinterpretation of the underlying joint motion and/or stability.

Moreover, research has already been conducted, for example, into identifying patient phenotypes based on kinematic joint signals during activities of daily life (e.g. Zgolli et al. 2018, Kinematic data clustering for healthy knee gait characterization; Mezghani et al. 2021, Healthy Knee Kinematic Phenotypes Identification Based on a Clustering Data Analysis; Petersen 2021, Petersen et al. 2021, Patients with knee osteoarthritis can be divided into subgroups based on tibiofemoral joint kinematics of gait: an exploratory and dynamic radiostereometric study), each using complex methods to detect the presence of statistically significant differences between patient clusters/patient groups. Unfortunately, further analyses have shown that those patient groups are not consistent either, for example, when the possibility of differently aligned local frames of segments is taken into account. Another disadvantage of these methods is that complex statistical analyses have to be carried out for three or six time series (three translations or three rotations or both, i.e. both three translations and three rotations) to determine statistically significant differences (e.g. by statistical parameter mapping). Therefore, no reliable and consistent assessment currently exists.

### SUMMARY

The tasks and objectives of the present disclosure are therefore to avoid the disadvantages of the prior art and, in particular, to provide a standardization or normalization system and a standardization or normalization method for the standardization or normalization of biomechanical kinematics data. Based on the standardized biomechanical kinematics data, a subtask can be seen in drawing conclusions for a medical intervention, in particular with regard to parameters of a medical implant and its relation to the patient. In particular, one object of the invention is to provide a device and a method for standardizing or normalizing axis alignment of biomechanical kinematics data, in particular rotational kinematics data and/or translational kinematics data, with respect to a body joint of a patient. The kinematics data thus comprise the translational kinematics data and/or the rotational kinematics data between two movable bodies or segments.

A standardization system and standardization method for comparing kinematic patterns are provided that do not require any prior knowledge of the position/pose, i.e., the orientation and position, in particular the orientation/alignment, of the original underlying reference frames, but enables the alignment and/or positioning of the two frames for comparison by optimizing the value of a selected criterion. In other words, the present disclosure comprises a normalization or standardization method or specially adapted control units (similar to an algorithm) that makes kinematic data from two or more sources (such as different tests, different test persons, different motion capture technologies, different laboratories, and the like) comparable by processing the underlying kinematic data such that at least one selected parameter is optimized. Processing the at least two data sets (with a specific implementation of the optimization) allows a valid comparison of the resulting rotational data and translational data, respectively, because differences due to axis misalignments and axis malpositions, respectively, have been or can be corrected equivalently in the at least two data sets.

In other words, the present disclosure proposes an optimization of the alignment and/or positioning, i.e. in particular the location, of coordinate systems for the standardization of kinematics data. The proposed standardization system and standardization method are based on the fundamental understanding that methods for minimizing crosstalk require a decision on how to define the “correct” orientations and/or the “correct” positioning of the reference frame. These decisions, which can also be regarded as underlying assumptions, are reflected in the choice of the target function(s) to be optimized (or minimized).

In the present case, an approach is proposed that solves this problem by providing a method or adapted control unit in which one or more specific (optimization) parameters or a target function are selected and then each data set (of kinematics data) is processed accordingly to obtain the rotations and/or translations resulting from the optimization described. Specifically, the processing involves the use of (mathematical) optimization algorithms to determine the orientations and/or positions of the segment reference frames that optimize a previously defined target function. These resulting angular curves (courses of standardized kinematic angles) and/or translational curves (courses of standardized kinematic translations) are then used for comparison and can be recorded for this purpose. The validity of the resulting comparison does not depend on the exact choice of the parameters to be optimized or the target function(s). One can favor a set of parameters or target functions that result, for example, in waveforms that are clinically easy to interpret, but the comparison does not lose validity if this is not the case.

In particular, a device is proposed as a standardization system for standardizing an axis alignment of rotational kinematics data and/or an axis position of translational kinematics data, in particular an axis position/axis pose, with respect to the body joint of a patient, comprising: a number of sensors for detecting and/or recording kinematics data sets and a computer to which the sensors are or can be connected for data transmission, the computer being adapted and designed to standardize orientation from the rotational data resulting from the recorded kinematics data sets, and/or the position of the tibial and femoral coordinate systems from the resulting translational data with the aid of a mathematical optimization method.

In particular, a (standardization) method is proposed for standardizing an axis alignment and/or an axis position of kinematics data, in particular of rotational kinematics data and/or translational kinematics data, with respect to the body joint of a patient by means of a device, preferably a standardization system according to the present disclosure, comprising: acquiring kinematics data sets relating to the body joint of a patient by means of a number of sensors; transmission to a computer; provision of rotational data and/or translational data resulting from the kinematics data sets and standardization of the orientation and/or position of tibial and femoral coordinate systems for the provided rotational data using a mathematical optimization method.

In other words, the present disclosure relates to a device and a method whereby the influence of the axial alignment on kinematics data (with respect to a patient joint, in particular a knee joint) as well as effects can be determined/reduced and an approach to normalization or standardization is provided.

In still quite different words, the present disclosure relates to a standard kinematics evaluation system for recording, standardizing and evaluating biomechanical kinematics data of the (selected) joint of a patient, comprising: at least one detecting device adapted to detect the biomechanical kinematics of a patient's joint (in particular kinematics rotations and kinematics translations) by way of sensors, record them and provide them in a computer-readable form, and a visual display device, in particular an operating room monitor, for visual output to a user. Furthermore, the standard kinematics evaluation system has a control unit that is specially adapted to: process the provided recorded kinematics of the patient and determine from this at least one progression of, in particular three, kinematics angles (as rotational angle graphs) and/or of, in particular three, translations of the (observed) joint vis-à-vis a first joint element with a first joint coordinate system and vis-à-vis a second joint element with a second joint coordinate system, determine, via a standardization unit that is adapted and designed for this purpose, a rotation vector or a rotation matrix for adjusting an orientation of the first joint coordinate system via a predetermined target optimization and/or a translation vector for adjusting a position of the first joint coordinate system via a predetermined target optimization, and determine a rotation vector or a rotation matrix for adjusting an orientation of the second joint coordinate system and/or determine a translation vector for adjusting a position of the second joint coordinate system via a predetermined target optimization in order to perform standardization, and output, via an evaluation unit which is adapted to carry out, based on the standardized kinematics angles and/or the standardized kinematics translations as motion data, an evaluation with regard to the joint for at least one predefined evaluation parameter, and output the evaluation via the display device.

In particular, a method for standardizing reference frame orientations and/or reference frame translations in biomechanical kinematics analysis using specific mathematical criteria is provided.

Very small changes in the alignment or positions of the underlying (reference) coordinate systems may cause large differences in the (characteristics of the) kinematics data. According to the disclosure, this effect can be taken account of when working out differences between gait patterns, in order to exclude the possibility that the differences found are based on such misalignments of the axes.

Examples of standardization scenarios are the post-processing of gait laboratory data (for standardization purposes) as well as the pre-processing of known kinematic gait patterns for comparison (for harmonization purposes).

According to a further, second aspect of the present disclosure, a standard kinematics evaluation system is provided for detecting, standardizing and evaluating biomechanical kinematics data of a patient's joint and an associated method which evaluates (characterizes) a laxity and/or stability and/or function of a human body joint, in particular a knee, in a normalized (and thus standardized) manner, the system or method being based on a mathematically predetermined target optimization which optimizes and thus normalizes a reference frame according to the present disclosure with respect to a position and/or orientation (reference frame alignment method). In this way, consistent and comparable measurements can be achieved. In particular, this system and method utilize the acquisition of kinematics data over a period of time or at several points in time, which are stored accordingly, and a comparison unit can be adapted to detect changes over time. Particular attention is thus paid in particular to the detection of kinematic changes over time.

The standard kinematics evaluation system according to the second aspect may in particular comprise optical and/or inertial sensors that are attached or attachable to the (relevant) joint segments (i.e., joint segments adjacent to the joint to be examined), in particular releasably fixed or fixable, for example, by means of a Velcro fastener, an adhesive or a band, and are adapted to acquire spatial or inertial data suitable for characterizing the motions of the joint segments during a series of activity cycles, whether passively through movement of a joint by a healthcare professional, or actively, such as walking or running. The acquired (raw) data from the sensors are forwarded to a control unit, which processes these data on the basis of a biomechanical model in order to calculate the corresponding kinematic signals of the joints (in particular of the joint segments that are part of the joint). A reference frame alignment method based on mathematical optimization of predetermined statistical parameters, in particular according to the present disclosure, can be used to characterize those kinematic signals and to (re)align/orient and/or (re)position the respective local joint segment coordinate frames to satisfy a predetermined underlying target function.

This enables consistent and reliable standardization of kinematic measurements for valid data comparison, in particular for an analysis with regard to a point in time of interest, be it current, historical or future. This reliable quantification of joint laxity and/or joint stability and/or joint function is particularly advantageous for determining a change, in particular a deterioration, of a joint and thus an optimal or best time for surgical intervention or for evaluating the course of a therapeutic treatment, in particular the progress of physiotherapy and/or rehabilitation after surgery. Thus, in the present case, a technical application for quantifying joint laxity in human, animal or mechanical joints in particular is provided, which is preferably adapted to detect a change, in particular a deterioration and/or progression in human and/or animal joints, such as a knee, hip, shoulder, head, neck, elbow and the associated (tissue) soft parts, in particular ligaments, muscles, cartilage, over time, and to evaluate it on the basis of this detection. In particular, a control unit can be adapted to calculate a so-called optimization remainder and output it to a medical specialist. An evaluation of the optimization remainder provides a direct objective measure for the conformity of the joint/axis estimation with a perfect joint/hinge. Any value that deviates from zero provides a direct quantification of laxity within the joint, which is applied in clinical examination, evaluation and monitoring of joint laxity/instability.

According to a still further, third aspect of the present disclosure a phenotyping method is provided for distinguishing between kinematic phenotypes of a human joint on the basis of motion data in order to simplify the choice of treatment, for example, the type of implant, the position of the implant and/or the alignment (orientation) of the implant relative to the affected joint segments, prior to surgery (for example, in the case of a total knee endoprosthesis).

According to this phenotyping method, the center of rotation of the first joint segment, in particular of the thigh, in a first step can be defined as the new origin of the frame (in particular after normalization according to the present disclosure as a second step) relative to an original (anatomical) first joint segment, in particular of the thigh. This new position of the first joint segment (e.g. the femur) enables the characterization of the joint kinematics (in particular of the knee joint) and individualized treatment recommendations, for example, by determining the specific implant design and/or the position and/or orientation that best suits a particular phenotype/patient, thereby optimizing patient satisfaction and the service life/durability of the implant.

This third aspect is intended to facilitate consistent identification of test person phenotypes based on kinematic signals, especially by simplifying the analysis of three or six complete time series (three translations or three rotations or both three translations and rotations) over one motion-loading cycle to obtain the simple evaluation of a 3×1 vector representing the relative first joint segment (in particular the femoral) frame center after standardization of the kinematic data set with a so-called REFRAME. With this phenotyping method, a virtual femoral center of rotation can be determined using a suitable kinematics definition (translations of the femur expressed in a tibia frame and any rotation definition) and suitable optimization criteria, in particular a minimization of the root mean square error (RMS) of all three translation components. This virtual femoral center of rotation is directly related to the characteristic movements of common implant types, such as, in particular, a medially stabilized (MS) implantation type with a medial center of rotation, a laterally stabilized (LS) implantation type with a lateralized center of rotation, or a posteriorly stabilized (PS) implantation type with a center of rotation located further distally. In particular, the phenotyping method may comprise a further step of determining a virtual femoral center of rotation, and based on the femoral center of rotation, selecting an implantation type, in particular determining the implantation type from the quantity consisting of the LS implantation type, the MS implantation type and the PS implantation type. In particular, a new joint coordinate system can be determined in one step according to the present disclosure, and it can be determined to which coordinate system of MS, LS and PS this new joint coordinate system is closest. This closest COS then indicates the implant type. For example, if the joint coordinate system is closest to the LS-COS, it can be determined that an LS implant type is suitable.

Thus, according to the third aspect, a method and system are provided that evaluate knee kinematics prior to surgery to determine the implant type that optimally matches the needs, gait pattern, and motion profile of a particular patient in order to improve the treatment outcome and service life of the medical device. The disclosure according to the third aspect in particular may be part of a medical device for measuring joint kinematics in a clinical setting during the preparation of total knee endoprosthesis surgery, in particular part of a standard kinematics evaluation system according to the present disclosure. In particular, the (same) method can be used to determine an actual hip pivot from gait data including an anatomical reference in the pelvis. In particular, it is important that also the orientation of the axes is indicated, so that a consistent interpretation of joint motion without crosstalk between the axes is possible. This allows the (correct) comparison of new motion data with historical motion data. In particular, the joint centers can be determined in one step for the evaluation of a knee adduction moment. It should be noted additionally that this approach not only applies to joints and medical applications, but also to other areas of application in which motion patterns from different sources or moving objects are clustered (or should be clustered). In particular, this method can also considerably simplify a comparison of motion patterns, since less effort is required for the correct arrangement of the coordinate frames. In particular, a rough (initial) placement is carried out in one step and in a subsequent step, the result is calculated using REFRAME, in particular according to the standard kinematics evaluation method according to the present disclosure.

Advantageous embodiments are explained in particular below.

According to one embodiment the at least one acquisition device may have at least one acceleration sensor and/or one gyro sensor, in particular an inertial measurement unit (IMU), as sensor, and the acquisition device can be adapted to be attached to the patient, e.g. on an extremity of the patient, in the region of the joint, in particular on the first joint element and/or the second joint element.

According to a further preferred embodiment, the at least one acquisition device may comprise at least one optical acquisition unit as sensor system, in particular a 3D acquisition unit, preferably a stereo camera, which captures the biomechanical kinematics of the patient's joint, preferably by way of optical markers, in particular passive infrared markers which are attached to the patient in the region of the joint, preferably at least to the first joint element and/or the second joint element.

Preferably, a minimization of a variance, a standard deviation, a square error and/or a statistical error of the regression of one or more kinematic parameters, in particular of one or more kinematic angles and/or of one or more kinematic translations, can be determined as the predetermined target optimization. Hence, of three kinematic angles in particular preferably two kinematic angles can be determined, in which, especially over exactly one cycle of joint motion, in particular a step cycle, the minimization of a variance or of a square error is determined as a target function and a change in the orientation of the first joint coordinate system and of the second joint coordinate system is defined as a target and a result, respectively, to be determined. Hence, the control unit calculates a contortion/re-orientation of the first local joint coordinate system about its origin and a contortion/re-orientation of the second local joint coordinate system about its origin, in which one, two or three of three kinematic angles are regarded and in these, for example, a surface of the graph of the corresponding kinematic angle is minimized approximately around its zero line. In other words, a variance, a standard deviation, a square error or a statistical error of the regression of one or more kinematic angles can be a selected parameter.—Model that uses the variance, the standard deviation, the mean square error, the root mean square error or the statistical error of the regression of one or more kinematic angles as criteria for minimizing crosstalk.

In particular, the standard kinematics evaluation system can be adapted to capture a knee joint with a tibial coordinate system as the first joint coordinate system (tibial KOS), and a femoral coordinate system (femoral KOS) as the second joint coordinate system and (by means of a gait analysis) determine a progression of the three kinematic angles of flexion, adduction and internal rotation and/or a progression of the three kinematic translations, in particular in the case of precisely a single step cycle, and the control unit can be adapted in particular to output, as an evaluation, a determination of the gait type and/or a selection of the size of a knee endoprosthesis (knee implant) and/or an orientation of a knee endoprosthesis as evaluation parameters.

Preferably, the predetermined target optimization can be defined as a minimization of a root-mean-square error (RMSE) (of the graph) of the kinematics angle of adduction and/or (the graph) of the kinematics angle of internal rotation. A minimization of a root-mean-square error (RMSE) is a particularly suitable target optimization, since in this way individual deflections are less strongly weighted and possible errors can be minimized.

In particular, an RMSE (Root Mean Square Error) can be selected as a measure of the deviations of the curves of varus/valgus and internal/external rotation. In particular, these can be reduced from 0.79±0.300 to 0.29±0.30° by rotating the coordinate systems around 3.32±1.24° around the associated screw axis.

Preferably, the predetermined target optimization can be defined as a minimization of a variance (of the graph) of the kinematic angle of adduction and/or (the graph) of the kinematic angle of internal rotation. When minimizing a variance, the graph of the corresponding kinematic angle may show an offset with respect to the zero line, but the graph should be as smooth as possible, i.e. flat with respect to the “new, offset zero line”.

According to one embodiment, the control unit can have a memory unit including a database with an assignment of standardized kinematic angles and/or standardized kinematic translations to evaluation parameters, in particular an assignment of the standardized kinematic angles to a size and alignment of a knee endoprosthesis. In this way, depending on the selected evaluation parameter (to some extent from a column to be selected on the evaluation side), an optimal matching of standardized kinematics angles to the corresponding evaluation parameters can be found.

According to a further embodiment, the control unit can be trained as an artificial intelligence system by way of a training data set with standardized kinematic angles and/or kinematic translations as input, and an output to an endoprosthesis of the patient's joint, so that the control unit is adapted to output an evaluation for an endoprosthesis, in particular for a size and alignment, when new biomechanical kinematics of a patient is detected via standardized kinematic angles and/or standardized kinematic translations by means of an artificial intelligence method. If standardized kinematics data in form of standardized kinematics angles for patients in general are available through the present disclosure, then this standardization can used for performing an evaluation by means of artificial intelligence and a trained system, respectively. This differs from the prior art, in which possible small errors lead to disproportionately large deviations and therefore make an evaluation no longer possible.

In particular, the control unit can be adapted to determine the kinematics angles by means of the recorded biomechanical kinematics of the patient, in particular when using an inertial measurement unit (IMU) as a sensor based on the accelerations and angular velocities to calculate three kinematics angles. In general, an IMU has an acceleration sensor for acquiring acceleration in three directions and a gyroscope for determining angular velocities.

In particular, kinematics data sets for level walking on an (established) knee joint simulator (VIVO, AMTI, Watertown, MA) can be available or entered in the standard kinematics evaluation system and recorded simultaneously with a number of sensors, preferably acceleration sensors and/or optical sensors, in particular at least one initial measurement unit.

In particular, the sensors of the detecting device can be arranged preferably in/on a knee cuff and can thus, for example, be temporarily attached and positioned on the knee in the manner of a bandage.

Moreover, the sensors are preferably connected to the control unit (such as a specially adapted computer system) or coupled to it for data transmission. For the resulting kinematics angle/rotation data, the orientation of the tibial and femoral coordinate systems is then normalized or standardized by means of an optimization (a mathematical optimization method).

With regard to a standard kinematics evaluation method of detecting, standardizing and evaluating biomechanical kinematics data of a (selected) joint of a patient, in particular in a standard kinematics evaluation system according to the present disclosure, the objects of the present disclosure are solved in that it comprises the steps of: acquiring the biomechanical kinematics of a patient by means of sensors of a detecting device; determining, by a control unit on the basis of the provided kinematics of the patient, the progression of kinematics angles and/or kinematics translations of the joint vis-à-vis a first joint element with a first joint coordinate system and a second joint element with a second joint coordinate system; determining, by a standardization unit of the control unit, via a predetermined target optimization, a rotation vector or a rotation matrix for adjusting an orientation of the first joint coordinate system and/or of the rotation vector or a rotation matrix for adjusting an orientation of the second joint coordinate system and/or, via a predetermined target optimization, a translation vector for adjusting a position of the first joint coordinate system and/or a translation vector for adjusting a position of the second joint coordinate system; standardizing the progression of the kinematics angles by adjusting the first and second joint coordinate systems using the correspondingly determined rotation vectors or rotation matrices, and/or the progression of the kinematics translations by adjusting the first and second joint coordinate systems by the correspondingly determined translation vectors; performing an evaluation of the joint with regard to a predefined evaluation parameter on the basis of the standardized kinematics angles and/or standardized kinematics translations; and outputting the evaluation by means of a display device.

In particular, the standardized kinematics evaluation method and the control unit, respectively, of the standardized kinematics evaluation system can be adapted to use a crosstalk minimization method as a target optimization.

Preferably, the standard kinematics evaluation method and the control unit of the standard kinematics evaluation system, respectively, can be adapted to optimize a reference frame orientation (reference coordinate system) in the knee kinematics.

Preferably, the standard kinematics evaluation method and the control unit of the standard kinematics evaluation system, respectively, can be adapted to provide a normalization (or standardization) of motion measurement systems. In particular, this includes systems based on optical markers and systems based on inertial measurement units (IMU).

In particular, the standard kinematics evaluation method can capture, standardize and evaluate a patient's knee joint as a joint, where a minimization of a root-mean-square error (RMSE) (of the graph) of the kinematics angle of adduction and/or (the graph) of the kinematics angle of internal rotation is defined as a predetermined target optimization.

According to a further embodiment of the standard kinematics evaluation method, a patient's knee joint can be recorded as a joint, standardized and evaluated, wherein a minimization of a variance of the kinematics angle of adduction and/or the kinematics angle of internal rotation is defined as a predetermined target optimization.

With regard to a computer-readable storage medium, the objects are fulfilled by the fact that it comprises instructions that, when executed by a computer, cause the computer to carry out the steps of the standard evaluation method.

### BRIEF DESCRIPTION OF THE DRAWINGS

The invention is explained in more detail below by means of figures, based on preferred embodiments.

FIG. 1 is a perspective view of a knee joint with a femoral coordinate system to illustrate an error in the prior art with regard to an analysis and evaluation that may occur when an orientation deviates;

FIG. 2 is a schematic view of a standard kinematics evaluation system according to a first preferred embodiment of the present disclosure;

FIGS. 3 and 4 show a schematic view explaining the three kinematics angles of the knee joint of the patient with corresponding standardization;

FIG. 5 shows a sequence of a standard kinematics evaluation method according to a first preferred embodiment of the present disclosure;

FIG. 6 shows a comparison of different sources as input to the standard kinematics evaluation system or a standard kinematics evaluation method;

FIGS. 6a to 6c show a schematic representation to illustrate the standardization of kinematics data of a patient with comparison to a kinematics simulation in which both the amplitude and the characteristics of the curves a to d change fundamentally due to the reorientation of the joint coordinate systems as reference systems;

FIG. 7 shows an overview of various acquisitions of kinematics data from patients, which were standardized according to the standard kinematics evaluation system or the standard kinematics evaluation method of the present disclosure regarding reorientations of the joint coordinate system;

FIG. 8 is a flow chart of a standard kinematics evaluation method according to a preferred embodiment; and

FIG. 9 shows a schematic representation of exemplary frame positions in a knee joint with typical centers of rotation for common implant types.

The figures are schematic in nature and are intended merely to help understanding the invention. Identical elements are provided with identical reference signs. The features of the various embodiments can be interchanged.

### DETAILED DESCRIPTION

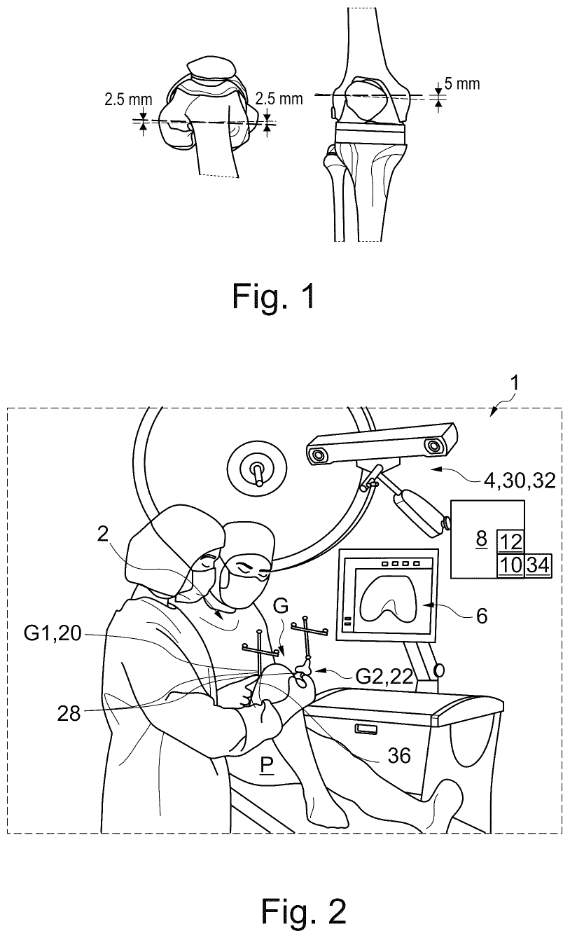

An example of an illustration of a concomitant deviation caused by a 3° rotation of the so-called flexion axis in a knee joint, is shown in FIG. 1. In a femur with a distance of 10 cm between the medial and lateral epicondyle, a 5 mm (total) AP or PD error in the detection of the bone landmarks already leads to a rotation of the reference system, i.e. the femoral coordinate system (FCS), by 3° around the Z or Z-axis. The maximum width of the corridors in adduction and internal rotation for ±3° of the rotations Ry and Rz in the orientation of the tibial and femoral coordinate system is already 14.5°. As described in the introduction to the present disclosure, incorrect alignment of the axes of the joint coordinate system results in crosstalk and corresponding crosstalk effects, so that, for example, a rotation in one plane is partially perceived as a rotation in other planes. A rotation of the femoral coordinate system about one axis therefore not only causes a change of a single kinematics angle, but also has an influence on the other two kinematics angles.

FIG. 2 schematically shows a standard kinematics evaluation system 1 according to a first preferred embodiment of the present disclosure. The standard kinematics evaluation system 1 is used to acquire, standardize and evaluate (in a comparing manner possible through standardization) biomechanical kinematics data of a knee joint G of a patient (P), as explained below.

The standard kinematics evaluation system 1 has at least one acquisition device 2 for detecting a biomechanical kinematics of the knee joint G of a patient P, which detects it via an optical sensor system 4 and makes it available to a control unit 8 in a computer-readable form. Furthermore, the standard kinematics evaluation system 1 has a display device, for example in the form of an operating room monitor, for displaying data relating to a surgical intervention to be performed on patient P and for outputting the evaluation results.

According to the present disclosure the control unit 8 is specifically adapted to process the detected kinematics of the patient P provided by the acquisition device 2 and to determine, on the basis of the kinematics data, a progression of three kinematics angles 14, 16, 18, as a rotation angle graph, (and/or three kinematics translations) of the knee joint G vis-à-vis a first joint element G1 with a first joint coordinate system 20 and vis-à-vis a second joint element G2 with a second joint coordinate system 22. In other words, for exactly one step cycle of a patient, the three kinematics angles 14, 16, 18 (and/or three kinematics translations) of a model joint are extracted from the abundance of kinematics data.

The standard kinematics evaluation system 1 thus is particularly adapted to acquire a knee joint G with a tibial coordinate system (tibial COS) as the first joint coordinate system 20, and a femoral coordinate system (femoral COS) as the second joint coordinate system 22, and to determine a progression of the three kinematics angles 14, 16, 18, i.e. a flexion 14, an adduction 16 and an internal rotation 18. 22 in exactly one single step cycle.

In order to be able to create a comparison of kinematic analyses of different patients (as well as simulations) that is as good as possible, or to be able to create a standardized analysis and associated evaluation of different acquisition methods, if possible (for example, temporally offset or using different acquisition methods, such as optical sensors versus attached sensors), of a single patient, the kinematics angle data must be standardized according to specific criteria in order to provide a uniform, standardized data acquisition system and a standardized data basis, respectively.

For this purpose, a standardization unit 10, which is adapted and designed to determine a rotation vector 24 for adjusting an orientation of the first joint coordinate system 20 and to determine a rotation vector 26 for adjusting an orientation of the second joint coordinate system, is used to perform a standardization via a predetermined target optimization.

The standard kinematics evaluation system 1 has an evaluation unit 12 adapted to carry out an evaluation with regard to the joint G for at least one predefined evaluation parameter on the basis of the standardized kinematics angles 14′, 16′, 18′ as motion data. In the present embodiment, a selection of the size of a knee endoprosthesis and an orientation of a knee endoprosthesis for the surgical intervention is output as an evaluation parameter. The visual output of the evaluation is performed by the display device 6, in particular by displaying preoperative CT data of the patient P, in which the simulated knee endoprosthesis is shown with the corresponding size and alignment.

The present standard kinematic evaluation system 1 allows robust and reliable evaluations to be performed since an error is minimized. A surgeon is even better supported in preparing for the surgical intervention and implanting the knee endoprosthesis, and the patient's safety is further increased.

For acquiring the kinematics of the patient, the at least one acquisition device 2 has several inertial measurement units 28 (hereinafter referred to as IMUs) as sensors, which are attached to two (detachable) knee cuffs 36 on the patient P's thigh and lower leg.

In addition to the IMUs, the standard kinematics evaluation system 1 has a further acquisition device 2, which has an optical recording unit 30 in the form of a stereo camera 32, which is aligned obliquely from above to a floor area in order to externally acquire a biomechanical kinematics of the joint G of the patient P. For this purpose, infrared markers are arranged on the knee cuffs 36 in addition to IMUs 28.

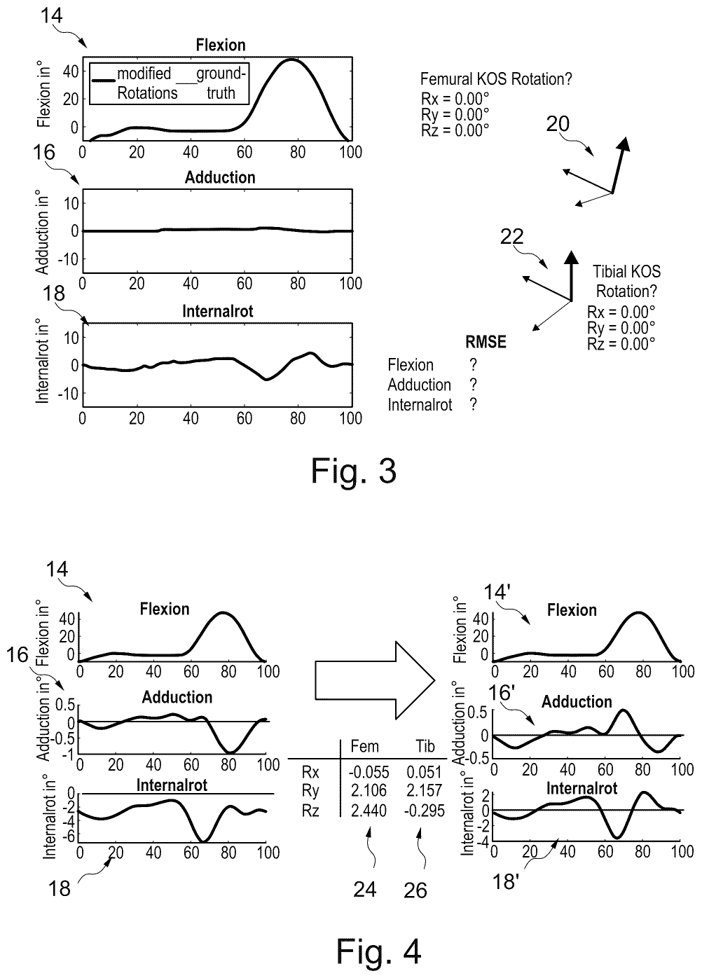

FIGS. 3 and 4 schematically visualize the three kinematics angles 14, 16, 18 and the joint coordinate systems 20, 22 of the standard kinematics evaluation system 1. The aim of the optimization in this embodiment is to minimize a root-mean-square error (RMSE) while varying an orientation of the joint coordinate systems 20, 22. The aim thus is to find a first rotation vector 24 and a second rotation vector 26 such that a root-mean-square error (RMSE) of both the adduction kinematics angle 16 and a root-mean-square error (RMSE) of the internal rotation angle 18 is minimized. In this example, the first femoral rotation vector 24 (Rx, Ry, Rz) is (−0.055; 2.106; 2.44) and the second tibial rotation vector 26 (Rx, Ry, Rz) is (0.051; 2.157; −0.295). The rotations are gimbal-mounted, i.e. first the rotation about the X-axis, then the rotation about the Y-axis and finally the rotation around the Z-axis (with the same origin).

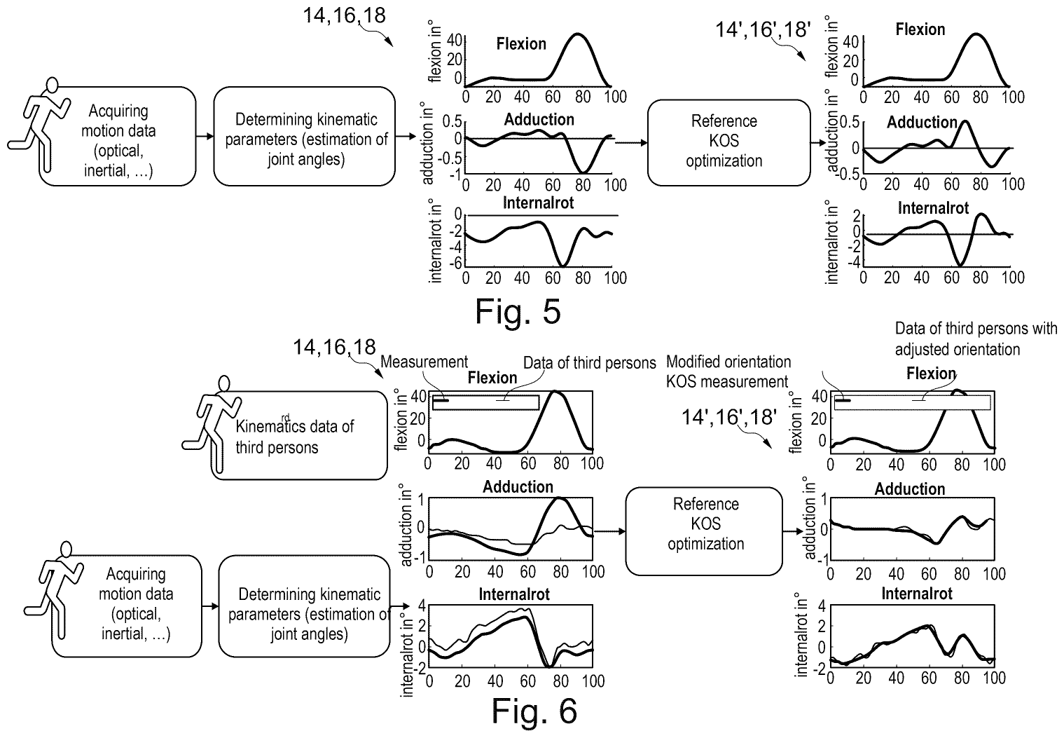

FIG. 5 shows the process of determining an orientation/alignment of the tibial and femoral coordinate systems (reference frames) by minimizing the root mean square error (RMSE) of adduction and internal rotation for standardization. In particular, an adduction/internal rotation of zero can be assumed during the entire cycle. Thus, kinematics data of the patient are first acquired including kinematics data for a joint of interest of the patient. In the next step, three kinematics angles 14, 16, 18 for a step cycle are extracted from the abundance of kinematic data, these kinematic angles 14, 16, 18 still being prone to error and a slight deviation in the alignment of the tibial or femoral coordinate system 20, 22 having significant influence on the progression of the kinematics angles.

The next step is target optimization with regard to minimizing a root-mean-square error (RMSE) of the adduction angle and the internal rotation angle, i.e. two of the three kinematic angles, the calculation of the rotation vectors, the application to the coordinate systems, the newly oriented coordinate systems now forming the reference systems for the femur and the tibia, and thus the standardization of the kinematics angles 14′, 16′ 18′ and their progressions, respectively, which changed accordingly due to the reorientation of the coordinate systems 20, 22.

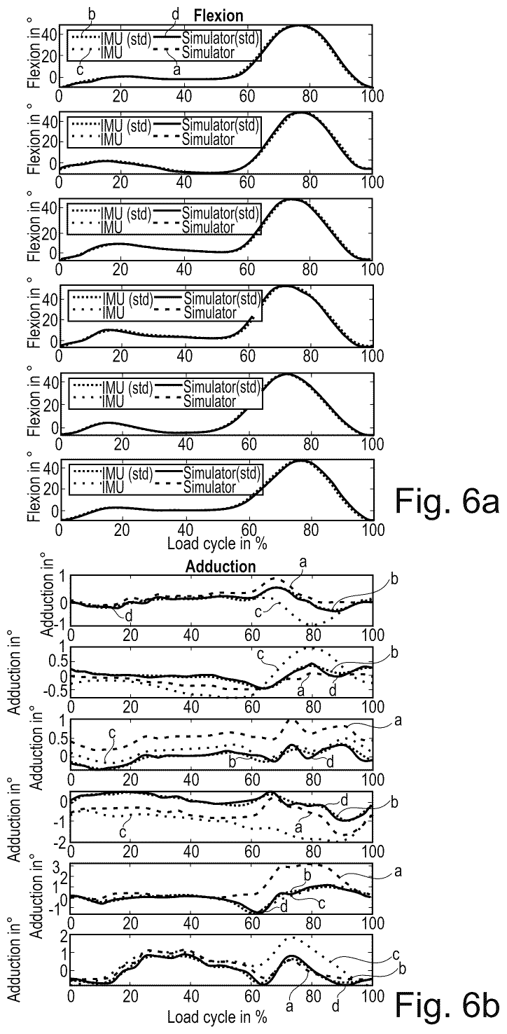

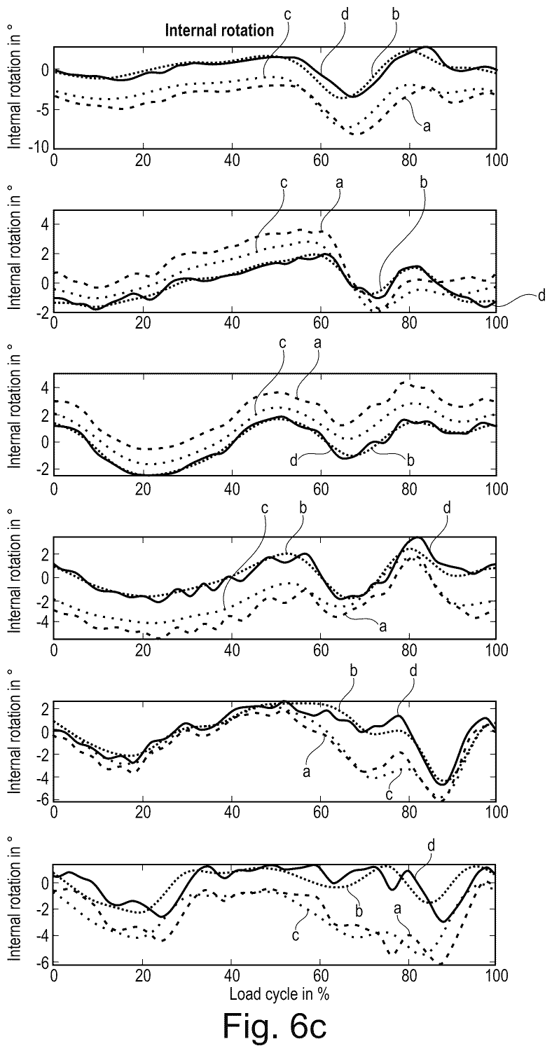

FIG. 6 shows a standardization of the orientations of the reference system when measuring the knee kinematics with a comparative data set of the kinematics of another patient, in particular of six patients. The standardization now allows data from different data sets, different acquiring methods, etc. to be compared with each other. Only through the standardization can it be recognized, for example, that only an error in the alignment causes non-comparable kinematics data and the error is corrected accordingly by the standardization. The standard kinematic evaluation system 1 is used to standardize reference frame orientations from measurements with different data sources in order to make them comparable.

FIGS. 6a to 6c show a schematic view of several recordings of kinematics angles of flexion 14, adduction 16 and internal rotation 18 through two different acquisition devices, optical and IMU. It can be discerned immediately that through standardization, the previously different kinematics angles 14, 16, 18 are now aligned and represent standardized kinematics angles 14′, 16′ and 18′, which can now be used by the standard kinematics evaluation system 1 for evaluation This also is plausible since patient P has a single step cycle that nevertheless has to be reproduced in the same way using different acquiring methods.

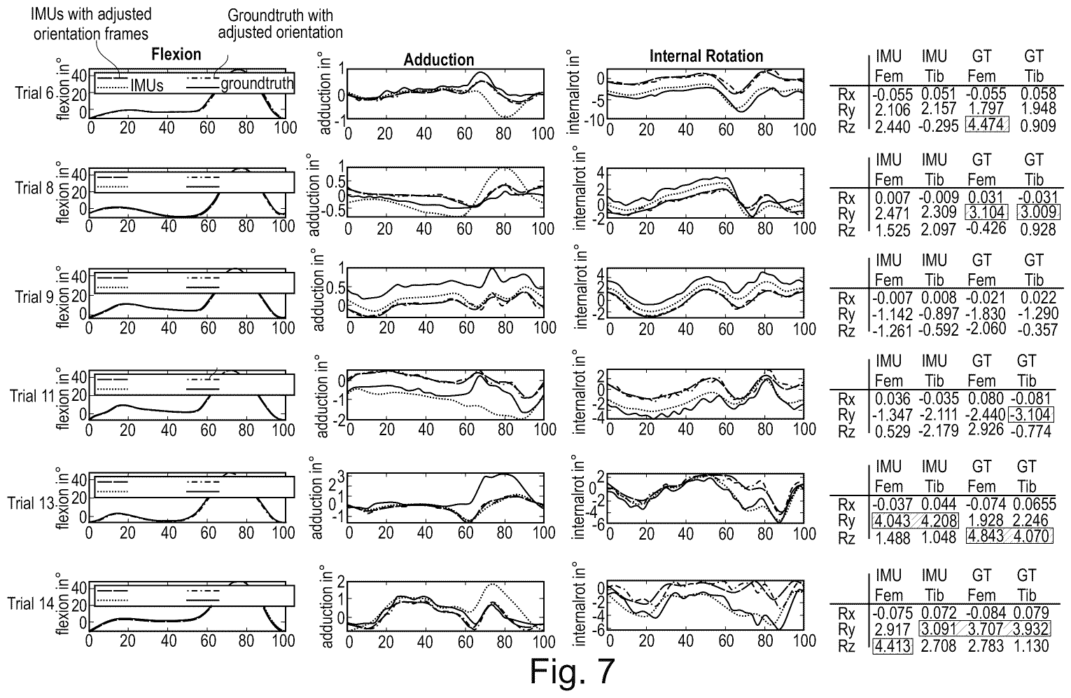

FIG. 7 shows a schematic view of different series of recorded kinematics, with and without the corresponding standardization through the standard kinematics evaluation system 1. Here, too, it is to be discerned that, when adapted, the curves lie even better on top of each other after standardization.

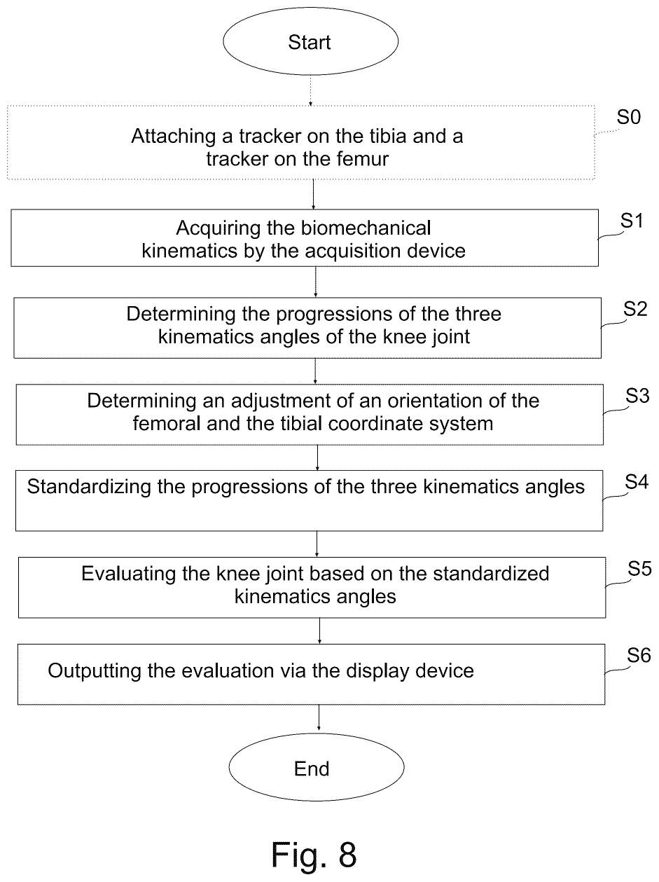

FIG. 8 shows a flow chart of a standard kinematics evaluation method according to a preferred embodiment of the present disclosure. The standard kinematics evaluation method for acquiring, standardizing and evaluating biomechanical kinematics data of a knee joint G of a patient P comprises the following steps.

In a first step S1, a biomechanical kinematics of a patient P is acquired by means of sensors 4 of an acquisition device 2. This can be done, in particular, using a stereo camera and optical markers or sensors attached to the patient.

In a subsequent second step S2, a control unit 8 determines, on the basis of the kinematics of the patient P provided, a progression of three kinematics angles 14, 16, 18 of the knee joint G relative to a first joint element G1 (femur) with a first joint coordinate system 20 and a second joint element G2 (tibia) with a second joint coordinate system 22.

This is followed by step S3: determining, through a standardization unit 10 of the control unit 8, via a predetermined target optimization, a rotation vector 24 for adjusting an orientation of the first joint coordinate system 20 and a rotation vector 26 for adjusting an orientation of the second joint coordinate system 22.

After the two rotation vectors 24, 26 have been found, step S4 of standardizing the progression of the kinematics angles 14,′ 16′, 18′ is carried out by adjusting the first and second joint coordinate systems 20, 22 by the correspondingly determined rotation vectors 24, 26. Thus, the coordinate systems are rotated according to the rotation vectors and their orientation is adjusted (and/or the translation vectors are shifted and their positions are adjusted). The progressions of the original kinematics angles 14, 16, 18 (and/or kinematics translations) likewise are adapted and become the standardized progressions of the kinematics angles 14′, 16′ and 18′ (and/or kinematics translations).

In a final step S5, an evaluation of the knee joint is carried out with regard to a predefined evaluation parameter based on the standardized kinematics angles. In particular, a classification of a gait is determined as an evaluation parameter and output in a final step S6 by a display device 4.

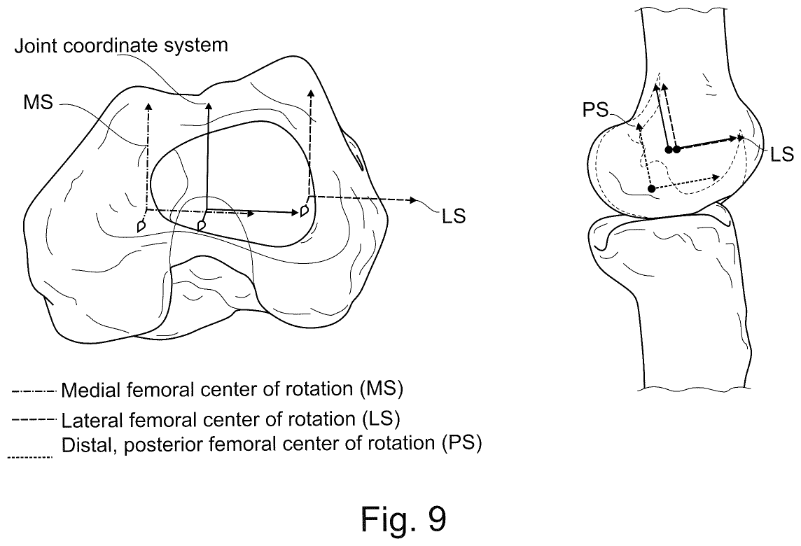

FIG. 9 shows examples of different frame positions and thus also coordinate systems in a knee joint. Shown in this are typical centers of rotation for common implant types, namely those of an MS implant type, an LS implant type and a PS implant type.

In particular, a standard kinematics evaluation method for acquiring, standardizing and evaluating biomechanical kinematics data of a joint G of a patient P can be performed in a standard kinematics evaluation system 1 according to the present disclosure in order to determine a translation vector and to thereby define a virtual position of the new joint coordinate system as a frame. After that, the phenotyping method according to the third aspect of the present disclosure can be performed in a further step. For example, if the new joint coordinate system that is closest to the MS coordinate system (COS) is determined for the joint, it can be determined that an MS implant type is suitable. However, if a new joint coordinate system is determined that is closest to the LS COS, it can be determined that an LS implant type is suitable. Finally, if a new joint coordinate system is determined that is closest to the PS COS, it can be determined that a PS implant type is suitable.

### LIST OF REFERENCE SIGNS

1 Standard kinematics evaluation system
2 Acquisition device
4 Sensor system
6 Display device
8 Control unit
10 Standardization unit
12 Evaluation unit
14 Flexion kinematics angle
14′ Standardized flexion kinematics angle
16 Adduction kinematics angle
16′ Standardized adduction kinematics angle
18 Internal rotation kinematics angle
18′ Standardized internal rotation kinematics angle
20 First joint coordinate system
22 Second joint coordinate system
24 First rotation vector
26 Second rotation vector
28 Inertial measurement unit (IMU)
30 Optical recording unit
32 Stereo camera
34 Storage unit
36 Knee cuff
G Joint
G1 First joint element
G2 Second joint element
P Patient
S1 Step of acquiring biomechanical kinematics using an acquisition device
S2 Step of determining a progression of kinematics angles
S3 Step of determining adjustment orientation
S4 Step of standardizing progression of kinematics angles
S5 Step of evaluating joint
S6 Step of outputting evaluation

1.-14. (canceled)

15. A standard kinematics evaluation system for acquiring, standardizing and evaluating biomechanical kinematics data of a joint of a patient, the standard kinematics evaluation system comprising:
at least one acquisition device adapted to acquire a biomechanical kinematics of the joint with a sensor system and to provide the biomechanical kinematics of the joint in a computer-readable manner;
a visual display device for visual output to a user;
a control unit;
a standardization unit; and
an evaluation unit,
the control unit adapted to:
process the biomechanical kinematics of the joint and calculate at least one progression of at least one kinematics angle and/or of at least one kinematics translation of the joint with respect to a first joint element having a first joint coordinate system and with respect to a second joint element having a second joint coordinate system,
determine, via the standardization unit, a rotation vector or a rotation matrix for adjusting an orientation of the first joint coordinate system, and/or determine a rotation vector or a rotation matrix for adjusting an orientation of the second joint coordinate system via a predetermined target optimization in order to perform a standardization, and/or determine a translation vector for adjustment of a position of the first joint coordinate system and/or determine a translation vector for adjustment of a position of the second joint coordinate system via a predetermined target optimization in order to perform a standardization, and
perform, via the evaluation unit, an evaluation with regard to the joint for at least one predefined evaluation parameter based on a standardized kinematics angle and/or a standardized kinematics translation as motion data, and output the evaluation via the visual display device.

16. The standard kinematics evaluation system according to claim 15, wherein the sensor system comprises at least one acceleration sensor and/or one gyro sensor, the at least one acquisition device being attachable to the patient in a region of the joint.

17. The standard kinematics evaluation system according to claim 15, wherein the at least one acquisition device includes at least one optical recording unit as the sensor system, the at least one optical recording unit configured to acquire the biomechanical kinematics of the joint of the patient.

18. The standard kinematics evaluation system according to claim 15, wherein a minimization of a variance, a standard deviation, a square error and/or a statistical error of a regression of the at least one kinematics angle is determined as a predetermined target optimization.

19. The standard kinematics evaluation system according to claim 15, wherein:
the at least one kinematics angle comprises a kinematics angle of a flexion, a kinematics angle of an adduction, and a kinematics angle of an internal rotation; and
the standard kinematics evaluation system is adapted to acquire a knee joint with a tibia coordinate system as a first joint coordinate system and a femur coordinate system as a second joint coordinate system and to determine a progression of the kinematics angle of the flexion, the kinematics angle of the adduction, and the kinematics angle of the internal rotation.

20. The standard kinematics evaluation system according to claim 19, wherein the control unit is adapted to output, as an evaluation, a determination of a gait type and/or a selection of a size of a knee endoprosthesis and/or an orientation of a knee endoprosthesis as an evaluation parameter.

21. The standard kinematics evaluation system according to claim 19, wherein a minimization of a root-mean-square error of the kinematics angle of the adduction and/or of the kinematics angle of the internal rotation is defined as a predetermined target optimization.

22. The standard kinematics evaluation system according to claim 19, wherein a minimization of a variance of the kinematics angle of the adduction and/or of the kinematics angle of the internal rotation is defined as a predetermined target optimization.

23. The standard kinematics evaluation system according to claim 15, wherein the control unit has a storage unit that includes a database with an assignment of standardized kinematics angles to evaluation parameters.

24. The standard kinematics evaluation system according to claim 23, wherein, the standardized kinematics angles are assigned to a size and an alignment of a knee endoprosthesis.

25. The standard kinematics evaluation system according to claim 15, wherein the control unit is trained as an artificial intelligence system by way of a training data set with standardized kinematics angles and/or standardized kinematics translations as input, and an output regarding an endoprosthesis of the joint of the patient, so that the control unit is adapted to output an evaluation for the endoprosthesis when a new biomechanical kinematics of a patient is acquired through standardized kinematics angles and/or standardized kinematics translations by an artificial intelligence method.

26. The standard kinematics evaluation system according to claim 25, wherein the evaluation comprises a size and an alignment for the endoprosthesis.

27. The standard kinematics evaluation system according to claim 15, wherein the control unit is adapted to determine kinematics angles based on the biomechanical kinematics of the patient and/or calculate kinematics translations.

28. A standard kinematics evaluation method for acquiring, standardizing and evaluating biomechanical kinematics data of a joint of a patient, the standard kinematics evaluation method comprising the steps of:
acquiring biomechanical kinematics of the joint of the patient with a sensor system of an acquisition device;
determining, by a control unit, based on the biomechanical kinematics of the joint, a progression of kinematics angles and/or kinematics translations of the joint relative to a first joint element with a first joint coordinate system, and a second joint element with a second joint coordinate system;
determining, by a standardization unit of the control unit, via a predetermined target optimization, a first rotation vector or a first rotation matrix for adjustment of an orientation of the first joint coordinate system and/or a second rotation vector or a second rotation matrix for adjustment of an orientation of the second joint coordinate system, and/or determining, via the predetermined target optimization, a first translation vector for adjustment of a position of the first joint coordinate system and/or determining a second translation vector for adjustment of a position of the second joint coordinate system;
standardizing the progression of the kinematics angles through adjustment of the first joint coordinate system and the second joint coordinate system by the first rotation vector and the second rotation vector, respectively, or the first rotation matrix and the second rotation matrix, respectively, and/or of the progression of the kinematics translations through adjustment of the first joint coordinate system and the second joint coordinate system by the first rotation vector and the second rotation vector, respectively;
performing an evaluation of the joint with respect to a predefined evaluation parameter based on standardized kinematics angles and/or standardized kinematics translations; and
outputting the evaluation with a display device.

29. The standard kinematics evaluation method according to claim 28, wherein:
the joint of the patient is a knee joint,
the standard kinematics evaluation method acquires, standardizes and evaluates the knee joint, and
a minimization of a root mean square error of a kinematics angle of adduction, and/or of a kinematics angle of internal rotation of a femur is defined, as the predetermined target optimization, to determine an adjustment of the orientation and/or position.

30. The standard kinematics evaluation method according to claim 28, wherein:
the joint of the patient is a knee joint,
the standard kinematics evaluation method acquires, standardizes and evaluates the knee joint, and
a minimization of a variance of a kinematics angle of adduction and/or of a kinematics angle of internal rotation is defined, as the predetermined target optimization, to determine an adjustment of the orientation and/or position.

31. A computer-readable storage medium comprising instructions that, when executed by a computer, cause the computer to carry out the standard kinematics evaluation method according to claim 28.
